(19) United States
(12) Patent Application Publication
(10) Pub. No.: US 2023/0414575 A1
(43) Pub. Date: Dec. 28, 2023

(54) MK2 ACTIVATING COMPOUNDS FOR USE IN TREATING VASCULAR LEAK AND ENDOTHELIAL BARRIER DISORDERS

(71) Applicant: Akttyva Therapeutics, Inc., Eastham, MA (US)

(72) Inventors: Katya Tsaioun, Eastham, MA (US); Usamah Kayyali, Belmont, MA (US); Bruno Villoutreix, Vincennes (FR)

(21) Appl. No.: 18/036,713

(22) PCT Filed: Nov. 12, 2021
(86) PCT No.: PCT/US2021/059191

§ 371 Date: May 12, 2023

Related U.S. Application Data
(60) Provisional application No. 63/112,878, filed on Nov. 12, 2020.

(51) Int. Cl. A61K 31/42; A61K 31/496; A61K 31/519; A61K 31/5415; A61K 31/4468; A61P 37/04

(52) U.S. Cl. CPC A61K 31/42; A61K 31/496; A61K 31/519; A61K 31/5415; A61K 31/4468; A61P 37/04

(57) ABSTRACT

Provided herein are MK2 activators, and compositions comprising an MK2 activator, for the treatment of a vascular disorder or an endothelial barrier disorder in a patient in need thereof, whereby the MK2 activator has a molecular weight below 1000 Da and binds to a binding pocket located on the C-lobe domain and C-terminal Regulatory Domain of MK2. Also provided are methods of treating a vascular disorder or an endothelial barrier disorder in a patient by administering to the patient an MK2 activator.

### CROSS-REFERENCE TO RELATED APPLICATION

This application claims the benefit of, and priority to, U.S. Provisional Application No. 63/112,878, filed on Nov. 12, 2020, the content of which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Nov. 12, 2021, is named AKY-001WO_ST25.txt and is 7,769 bytes in size.

### BACKGROUND

The vascular endothelium separates circulating fluid and inflammatory cells in blood from the surrounding tissues. Vascular leak typically occurs in response to wide-spread inflammatory processes, which may arise in response to a chronic disease, as well as in acute situations, in response to viral, bacterial or a direct toxins exposure. There are over sixty medical conditions that are caused by vascular leak, including edema and acute kidney injury, among others. For example, in the eye, vascular leak may lead to macular degeneration such as diabetic retinopathy; in the lung, vascular leak may lead to sepsis, acute lung injury (ALI) and acute respiratory distress syndrome (ARDS). ARDS is a life-threatening disorder in which the alveolocapillary permeability barrier in the lung becomes leaky, flooding the lungs with fluid and diminishing the lung's ability to serve its main function: to provide essential organs with oxygen. This leads to alveolar flooding, hypoxemia and respiratory failure. When respiratory viruses (e.g., influenza, coronaviruses, SARS, MERS) reach the lung, they indirectly disrupt the endothelial membrane in alveoli and, in the most severe manifestation of the disease, lead to ARDS. The severe acute respiratory syndrome coronavirus 2 (SARS-CoV-2), a highly transmissible and pathogenic coronavirus that emerged in late 2019 and has caused a pandemic of acute respiratory disease, named ‘coronavirus disease 2019’ (COVID-19), is acting in the same way. Although ALI/ARDS are leading causes of mortality in the intensive care unit even before COVID-19, no medical therapies exist to restore the underlying endothelial cell barrier function. Mechanical ventilation is a standard therapy to maintain adequate gas exchange during ARDS, but it can lead to the acceleration of inflammatory process and augment a pulmonary damage. Therefore, there remains an unmet need to develop novel therapeutic strategies for treating ARDS to combat the most severe manifestation of respiratory infections, and to treat other vascular leak disorders in patients.

### SUMMARY

In one aspect, provided herein are MK2 activators for treating a vascular disorder or an endothelial barrier disorder in a patient in need thereof, wherein the MK2 activator has a molecular weight of less than 1000 Da; and the MK2 activator binds to a binding pocket located on the C-lobe domain and C-terminal Regulatory Domain of MK2.

In certain embodiments, the C-lobe domain of MK2 comprises the amino acid sequence of SEQ ID NO: 2. In certain embodiments, the C-terminal Regulatory Domain of MK2 comprises the amino acid sequence of SEQ ID NO: 3.

In certain embodiments, the binding pocket located on the C-lobe domain and C-terminal Regulatory Domain of MK2 comprises the amino acid residues M275, K276, I279, V352, E354, E355, S358, A359, and T362. In certain embodiments, the binding pocket located on the C-lobe domain and C-terminal Regulatory Domain of MK2 comprises the amino acid residues Y260, Y264, S265, N266, H267, E285, F286, P287, N288, P289, E290, and V341. In certain embodiments, the binding pocket located on the C-lobe domain and C-terminal Regulatory Domain of MK2 comprises the amino acid residues Y260, Y264, S265, N266, H267, A270, I271, S272, P273, G274, M275, K276, R278, I279, E285, F286, P287, N288, P289, E290, V341, E347, R348, E350, D351, V352, E354, E355, S358, A359, and T362.

In certain embodiments, the binding pocket is located between amino acid residues Y260 and T362. In certain embodiments, the binding pocket is located between amino acid residues E290 and D351.

In certain embodiments, the MK2 activator binds to the binding pocket having an binding affinity of about −5 kcal or lower. In certain embodiments, the MK2 activator is selective for MK2 over other kinases.

In certain embodiments, the MK2 activator is selected from the group consisting of Sepimostat, Bifepramide, Capeserod, Bifeprofen, Lorcinadol, Sulfamazone, Altapizone, Zaldaride, Netupitant, Casopitant, Pipamazine, Saperconazole, Barmastine, Bgt-226, Imidocarb, Elopiprazole, Oxaflumazine, Flupentixol, Clocapramine, Paliperidone, Tiospirone, Sertindole, Perospirone, Ziprasidone, Picloxydine, Amg-548, Cep-32496, Gsk-1070916, Gsk-461364, Ly-2584702, Fludoxopone, Clodoxopone, Amg-900, Mk-6592, Mk-8033, Azd-1775, Mubritinib, Mocetinostat, Pf-3758309, R428, Raltitrexed, XL228, Azd3514, Elinogrel, Tak-901, Chir-265, Bafetinib, Ac-480, Cc-401, Nilotinib, Radotinib, Golvatinib, Abexinostat, Ast-487, Enmd-2076, Bms-833923, Draflazine, Tezosentan, Lometrexol, Cinuperone, Icopezil-maleate, Elbanizine, Glisindamide, Camicinal, Tegobuvir, Flumeridone, Lomitapide, Mazokalim, Losulazine, Peraquinsin, Leflunomide, Afacifenacin, Ag-13958, Lorpiprazole, Siponimod, Mespiperone, XL019, Bentipimine, Zosuquidar, Fasitibant, Amg-517, Ci-988, Boxidine, Glicaramide, Niaprazine, Isometamidium, Metofenazate, Trenizine, Flotrenizine, Difluanine, Ramatroban, Sonepiprazole, Tafamidis, Verlukast, Ropizine, Lotrifen, Mioflazine, Talotrexin, Tiflamizole, Trefentanil, Asapiprant, Lidoflazine, Ruzadolane, Preladenant, Medibazine, Delavirdine, Piflutixol, Eberconazole, Teneligliptin, Daltroban, Deracoxib, Relenopride, Avagacestat, and combinations thereof.

In certain embodiments, the MK2 activator is selected from the group consisting of Sepimostat, Bifepramide, Capeserod, Bifeprofen, Lorcinadol, Sulfamazone, Altapizone, Zaldaride, Netupitant, Casopitant, Pipamazine, Saperconazole, Barmastine, Bgt-226, Imidocarb, Elopiprazole, Oxaflumazine, Flupentixol, Clocapramine, Paliperidone, Tiospirone, Sertindole, Perospirone, Ziprasidone, Picloxydine, Ly-2584702, Fludoxopone, Clodoxopone, Mubritinib, Mocetinostat, Pf-3758309, Raltitrexed, Azd3514, Elinogrel, Ac-480, Abexinostat, Bms-833923, Draflazine, Tezosentan, Lometrexol, Cinuperone, Icopezil-maleate, Elbanizine, Glisindamide, Camicinal, Tegobuvir, Flumeridone, Lomitapide, Mazokalim, Losulazine, Peraquinsin, Afacifenacin, Ag-13958, Lorpiprazole, Siponimod, Mespiperone, Bentipimine, Zosuquidar, Fasitibant, Amg-517, Ci-988, Boxidine, Glicaramide, Niaprazine, Isometamidium, Metofenazate, Flotrenizine, Difluanine, Ramatroban, Sonepiprazole, Tafamidis, Verlukast, Ropizine, Lotrifen, Mioflazine, Talotrexin, Tiflamizole, Trefentanil, Asapiprant, Lidoflazine, Ruzadolane, Preladenant, Medibazine, Delavirdine, Piflutixol, Eberconazole, Teneligliptin, Daltroban, Deracoxib, Relenopride, Avagacestat, and combinations thereof.

In certain embodiments, the MK2 activator is selected from the group consisting of Abexinostat (PCI-24781), Ac-480 (BMS-599626), Adavosertib (MK-1775), Ag-13958, Amg-517, Amg-900, Asapiprant, Ast-487 (NVP-AST487), Avagacestat (BMS-708163), Azd3514, BAF312 (Siponimod), Bafetinib (INNO-406), Bemcentinib (R428), Bgt-226 (NVP-BGT226), Bms-833923, Cc-401 Hydrochloride, Cep-32496, Delavirdine (mesylate), Deracoxib, Dropropizine, Enmd-2076, Flupentixol dihydrochloride, Golvatinib, Gsk-1070916, Gsk-461364, Imidocarb dipropionate, Leflunomide, Levodropropizine, Lomitapide, Ly-2584702, MK2-AP (batch #2), Mocetinostat (MGCD0103), Mubritinib (TAK 165), Nafamostat Mesylate, Netupitant, Nilotinib (AMN-107), Paliperidone, Perospirone hydrochloride, Pf-3758309, Preladenant, Radotinib, RAF265 (Chir-265), Raltitrexed, Ramatroban, Tafamidis, Tak-901, Tegobuvir, Teneligliptin hydrobromide, XL019, XL228, Ziprasidone HCl, Zosuquidar (LY335979) 3HCl, and combinations thereof.

In certain embodiments, the MK2 activator is selected from the group consisting of Lomitapide, Perospirone, Abexinostat, AG-13958, Levodropropizine, Nilotinib, AMG-900, AMG-517, Delavirdin, Bgt-226, Mocetinostat, Tafamidis, XL019, Radotinib, Ziprasidone, Mubritinib, Ramatroban, Deracoxib, Raltitrexed, GSK461364, Adavosertib, Tegobuvir, and combinations thereof.

In certain embodiments, the MK2 activator is selected from the group consisting of Leflunomide, Netupitant, Paliperidone, Lomitapide, Perospirone hydrochloride, and combinations thereof.

In another aspect, provided herein are compositions for treating a vascular disorder or an endothelial barrier disorder comprising a therapeutically effective amount of an MK2 activator described herein.

In another aspect, provided herein are pharmaceutical compositions for the treatment of a vascular disorder or an endothelial barrier disorder comprising a therapeutically effective amount of an MK2 activator described herein and a pharmaceutically acceptable excipient.

In another aspect, provided herein are unit dosage forms for the treatment of a vascular disorder or an endothelial barrier disorder comprising a therapeutically effective amount of an MK2 activator described herein.

In another aspect, provided herein are methods for treating a vascular disorder or an endothelial barrier disorder in a patient in need thereof, the method generally comprising administering to the patient a therapeutically effective amount of an MK2 activator described herein.

In certain embodiments, the vascular disorder is a vascular leak disorder.

In certain embodiments, the endothelial barrier disorder is a microvascular barrier leak disorder. In certain embodiments, the microvascular barrier leak disorder is selected from the group consisting of inflammatory bowel disease, brain edema, acute lung injury, acute respiratory distress syndrome, and pulmonary edema.

In certain embodiments, the acute respiratory distress syndrome is associated with COVID-19. In certain embodiments, the acute respiratory distress syndrome is associated with a hyper-permeability of an endothelial barrier. In certain embodiments, the acute respiratory distress syndrome is associated with an infectious disease selected from the group consisting of middle east respiratory syndrome (MERS), severe acute respiratory syndrome (SARS), and pneumonia.

In certain embodiments, the acute lung injury is associated with COVID-19. In certain embodiments, the acute lung injury is associated with a hyper-permeability of endothelial barrier. In certain embodiments, the acute lung injury is associated with an infectious disease selected from the group consisting of middle east respiratory syndrome (MERS), severe acute respiratory syndrome (SARS), and pneumonia.

In certain embodiments, the endothelial barrier disorder is a blood vessel barrier leak disorder. In certain embodiments, the blood vessel barrier leak disorder is selected from the group consisting of atherosclerosis, hypertension, preeclampsia, and Kawasaki disease.

In certain embodiments, the endothelial barrier disorder is a fibroblast-related disorder. In certain embodiments, the fibroblast-related disorder is selected from the group consisting of wound healing, pulmonary fibrosis, liver fibrosis, vascular fibrosis, kidney fibrosis, and tissue remodeling.

In certain embodiments, the vascular disorder is a vascular barrier disorder. In certain embodiments, the vascular barrier disorder is a gastrointestinal disorder. In certain embodiments, the gastrointestinal disorder is associated with COVID-19. In certain embodiments, the gastrointestinal disorder associated with a hyper-permeability of endothelial barrier.

In another aspect, provided herein are methods of treating an acute respiratory distress syndrome in a patient in need thereof, the method generally comprising administering to the patient a therapeutically effective amount of an MK2 activator described herein. In certain embodiments, the acute respiratory distress syndrome is associated with COVID-19.

In another aspect, provided herein are methods of treating an acute lung injury in a patient in need thereof, the method generally comprising administering to the patient a therapeutically effective amount of an MK2 activator described herein. In certain embodiments, the acute lung injury is associated with COVID-19.

In certain embodiments, the MK2 activator is administered intravenously to the patient. In certain embodiments, the MK2 activator is administered orally to the patient. In certain embodiments, the MK2 activator is administered parentally to the patient. In certain embodiments, the patient is a human.

### BRIEF DESCRIPTION OF THE DRAWINGS

FIG. 1 depicts images of Western blots using anti-phospho HSP-27 and anti-HSP-27 antibodies against lysates from cells treated with and without MK2 activators.

FIG. 2 is a histogram showing the normalized phosphorylation signal from Western blots as seen with various putative MK2 binding activators.

FIG. 3 is a phospho-HSP-27 ELISA standard curve generated by directly analyzing the anti-phospho-HSP-27 antibody reagent.

### DETAILED DESCRIPTION

As generally described herein, the present disclosure provides methods of treating a disorder described herein, for example, a vascular disorder or an endothelial barrier disorder, in a patient in need thereof using an MK2 activator. Also provided herein are compositions and pharmaceutical compositions comprising a therapeutically effective amount of an MK2 activator, or a pharmaceutically acceptable salt thereof.

### MK2 Activators

Mitogen-activated protein kinase-activated protein kinase 2 (MK2 or MAPKAPK2), UniProt ID P49137, is a kinase belonging to the mitogen-activated protein kinase-activated protein kinase (MAPKAPK) family, a group of kinases activated by mitogen-activated protein kinase (MAPK) signaling. MK2 is activated by p38alpha and regulates the production of inflammatory cytokines. This kinase is regulated through direct phosphorylation by p38 MAP kinase. In conjunction with p38 MAP kinase, this kinase is known to be involved in many cellular processes including stress and inflammatory responses, nuclear export, gene expression regulation and cell proliferation.

In some embodiments, the MK2 protein is the full length protein or a functional fragment thereof. In certain embodiments, the MK2 protein comprises the amino acid sequence of SEQ ID NO: 1, as listed in Table 1. In certain embodiments, the MK2 protein comprises a functional fragment of the amino acid sequence of SEQ ID NO: 1, e.g., the catalytic domain. In certain embodiments, MK2 protein or functional fragment thereof comprises an amino acid comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 1.

The MK2 activators described herein can be useful in the treatment, prophylaxis, or reduction in the risk of a disorder described herein. For example, an MK2 activator described herein may be useful in treating a vascular disorder or an endothelial barrier disorder in a patient in need thereof. In certain embodiments, provided herein is an MK2 activator for the treatment of a vascular disorder. In certain embodiments, provided herein is an MK2 activator for the treatment of an endothelial barrier disorder.

In certain embodiments, the MK2 activator has a molecular weight of less than 500 Da, 550 Da, 600 Da, 650 Da, 700 Da, 750 Da, 800 Da, 850 Da, 900 Da, 950 Da, 1000 Da, 1050 Da, 1100 Da, 1150 Da, 1200 Da, 1250 Da, 1300 Da, 1350 Da, 1400 Da, 1450 Da, or 1500 Da. In some embodiments, the MK2 activator has a molecular weight of less than 1000 Da.

In certain embodiments, the MK2 activator binds to a binding pocket located on the C-lobe domain of MK2. In certain embodiments, the C-lobe domain of MK2 comprises the amino acid sequence of SEQ ID NO: 2, as listed in Table 1. In certain embodiments, the C-lobe domain of MK2 comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 2.

In certain embodiments, the MK2 activator binds to a binding pocket located on the C-terminal Regulatory Domain of MK2. In certain embodiments, the C-terminal Regulatory Domain of MK2 comprises the amino acid sequence of SEQ ID NO: 3, as listed in Table 1. In certain embodiments, the C-terminal Regulatory Domain of MK2 comprises an amino acid comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO: 3.

In certain embodiments, the MK2 activator binds to a binding pocket located on the C-lobe domain and C-terminal Regulatory Domain of MK2.

In various embodiments, provided herein are MK2 activators for treating a vascular disorder or an endothelial barrier disorder in a patient in need thereof, wherein the MK2 activator has a molecular weight of less than 1000 Da; and the MK2 activator binds to a binding pocket located on the C-lobe domain and C-terminal Regulatory Domain of MK2.

In certain embodiments, the binding pocket located on the C-lobe domain and C-terminal Regulatory Domain of MK2 comprises one or more amino acid residues (e.g., one amino acid, two amino acids, three amino acids, four amino acids, five amino acids, six amino acids, seven amino acids, eight amino acids, or nine amino acids) of SEQ ID NO: 1 selected from the group consisting of M275, K276, I279, V352, E354, E355, S358, A359, and T362. In certain embodiments, the binding pocket located on the C-lobe domain and C-terminal Regulatory Domain of MK2 comprises the amino acid residues M275, K276, I279, V352, E354, E355, S358, A359, and T362 of SEQ ID NO: 1.

In certain embodiments, the binding pocket located on the C-lobe domain and C-terminal Regulatory Domain of MK2 comprises one or more amino acid residues (e.g., one amino acid, two amino acids, three amino acids, four amino acids, five amino acids, six amino acids, seven amino acids, eight amino acids, nine amino acids, ten amino acids or more) of SEQ ID NO: 1 selected from the group consisting of Y260, Y264, S265, N266, H267, E285, F286, P287, N288, P289, E290, and V341 of SEQ ID NO: 1. In certain embodiments, the binding pocket located on the C-lobe domain and C-terminal Regulatory Domain of MK2 comprises the amino acid residues Y260, Y264, S265, N266, H267, E285, F286, P287, N288, P289, E290, and V341.

In certain embodiments, the binding pocket located on the C-lobe domain and C-terminal Regulatory Domain of MK2 comprises one or more amino acid residues (e.g., one amino acid, two amino acids, three amino acids, four amino acids, five amino acids, six amino acids, seven amino acids, eight amino acids, nine amino acids, ten amino acids or more) of SEQ ID NO: 1 selected from the group consisting of Y260, Y264, S265, N266, H267, A270, I271, S272, P273, G274, M275, K276, R278, I279, E285, F286, P287, N288, P289, E290, V341, E347, R348, E350, D351, V352, E354, E355, S358, A359, and T362 of SEQ ID NO: 1. In certain embodiments, the binding pocket located on the C-lobe domain and C-terminal Regulatory Domain of MK2 comprises the amino acid residues Y260, Y264, S265, N266, H267, A270, I271, S272, P273, G274, M275, K276, R278, I279, E285, F286, P287, N288, P289, E290, V341, E347, R348, E350, D351, V352, E354, E355, S358, A359, and T362.

In certain embodiments, the binding pocket is located between amino acid residues Y260 and T362 of SEQ ID NO: 1. In certain embodiments, the binding pocket is located between amino acid residues E290 and D351 of SEQ ID NO: 1.

TABLE 1

| SED ID | Amino Acid Sequence |
|---|---|
| SEQ ID NO: 1 | MLSNSQGQSPPVPFPAPAPPPQPPTPALPHPPAQPPPPPPQQFPQFHVKSG |
| | LQIKKNAIIDDYKVTSQVLGLGINGKVLQIFNKRTQEKFALKMLQDCPK |
| | ARREVELHWRASQCPHIVRIVDVYENLYAGRKCLLIVMECLDGGELFSR |
| | IQDRGDQAFTEREASEIMKSIGEAIQYLHSINIAHRDVKPENLLYTSKRPN |
| | AILKLTDFGFAKETTSHNSLTTPCYTPYYVAPEVLGPEKYDKSCDMWSL |
| | GVIMYILLCGYPPFYSNHGLAISPGMKTRIRMGQYEFPNPEWSEVSEEV |
| | KMLIRNLLKTEPTQRMTITEFMNHPWIMQSTKVPQTPLHTSRVLKEDKE |
| | RWEDVKEEMTSALATMRVDYEQIKIKKIEDASNPLLLKRRKKARALEA |
| | AALAH |
| SEQ ID NO: 2 | LDGGELFSRIQDRGDQAFTEREASEIMKSIGEAIQYLHSINIAHRDVKPEN |
| | LLYTSKRPNAILKLTDFGFAKETTSHNSLTTPCYTPYYVAPEVLGPEKYD |
| | KSCDMWSLGVIMYILLCGYPPFYSNHGLAISPGMKTRIRMGQYEFPNPE |
| | WSEVSEEVKMLIRNLLKTEPTQRMTITEFMNHPWIMQ |
| SEQ ID NO: 3 | STKVPQTPLHTSRVLKEDKERWEDVKEEMTSALATMRVDYEQIKIKKIE |
| | DASNPLLLKRRKKARALEAAALAH |

In certain embodiments, the MK2 activator binds to the binding pocket having an binding affinity of about −1 kcal or lower, about −1.5 kcal or lower, about −2 kcal or lower, about −2.5 kcal or lower, about −3 kcal or lower, about −3.5 kcal or lower, about −4 kcal or lower, about −4.5 kcal or lower, about −5 kcal or lower, about −5.5 kcal or lower, about −6 kcal or lower, about −6.5 kcal or lower, about −7 kcal or lower, about −7.5 kcal or lower, or about −8 kcal or lower. In certain embodiments, the MK2 activator binds to the binding pocket having an binding affinity of about −5 kcal or lower.

In certain embodiments, the MK2 activator is selective for MK2 over other kinases.

In certain embodiments, the MK2 activator is an MK2 activator selected from any compound set forth in Table 2, or a pharmaceutically acceptable salt thereof.

Examples of MK2 activators include, but are not limited to, Flumeridone, Zaldaride, Metofenazate, Ci-988, Ac-480, Camicinal, Netupitant, Casopitant, Elinogrel, Cinuperone, Siponimod, Fasitibant, Barmastine, Mocetinostat, Abexinostat, Bms-833923, Glicaramide, Afacifenacin, Mazokalim, Amg-517, Pf-03758309, Lomitapide, Sulfamazone, Raltitrexed, Mubritinib, Zosuquidar, Elopiprazole, Clocapramine, Capeserod, Oxaflumazine, Flupentixol, Paliperidone, Tiospirone, Sertindole, Perospirone, Ziprasidone, Icopezil-maleate, Losulazine, Lorpiprazole, Mespiperone, Niaprazine, Bifepramide, Peraquinsin, Bentipimine, Glisindamide, Draflazine, Bgt-226, Lometrexol, Isometamidium, Tegobuvir, Sepimostat, Saperconazole, Picloxydine, Azd3514, Boxidine, Altapizone, Flotrenizine, Difluanine, Bifeprofen, Lorcinadol, Pipamazine, Imidocarb, Ly-2584702, Fludoxopone, Clodoxopone, Elbanizine, Tezosentan, Ag-13958, gemcitabine, Ramatroban, Sonepiprazole, Tafamidis, Verlukast, Ropizine, Lotrifen, Mioflazine, Talotrexin, Tiflamizole, Trefentanil, Asapiprant, Lidoflazine, Ruzadolane, Preladenant, Medibazine, Delavirdine, Piflutixol, Eberconazole, Teneligliptin, Daltroban, Deracoxib, Relenopride, and Avagacestat. Other examples of MK2 activators include, but not limited to, Amg-548, Cep-32496 (Agerafenib), Gsk-1070916, Gsk-461364, Amg-900, Mk-6592, Mk-8033, Azd-1775 (Adavosertib), R428 (Bemcentinib), Xl-228, Tak-901, Chir-265, Bafetinib, Cc-401, Nilotinib, Radotinib, Golvatinib, Ast-487, Enmd-2076, Leflunomide, Xl-019, and Trenizine.

Other embodiments of MK2 activators of the present invention can be found in, for example, Lagarde et al, Oncotarget., 2018, 9(64), 32346-32361; Wishart et al, Nucleic Acids Res., 2018, 46(D1), D1074-D1082; Ursu et al, Nucleic Acids Res. 2019, 47(D1), D963-D970; Siramshetty et al, Nucleic Acids Res., 2018, 46(D1), D1137-D1143; and Mendez et al, Nucleic Acids Res., 2019, 47(D1), D930-D940, each of which are incorporated herein by reference in their entirety.

In certain embodiments, the MK2 activator is selected from the group consisting of Sepimostat, Bifepramide, Capeserod, Bifeprofen, Lorcinadol, Sulfamazone, Altapizone, Zaldaride, Netupitant, Casopitant, Pipamazine, Saperconazole, Barmastine, Bgt-226, Imidocarb, Elopiprazole, Oxaflumazine, Flupentixol, Clocapramine, Paliperidone, Tiospirone, Sertindole, Perospirone, Ziprasidone, Picloxydine, Amg-548, Cep-32496, Gsk-1070916, Gsk-461364, Ly-2584702, Fludoxopone, Clodoxopone, Amg-900, Mk-6592, Mk-8033, Azd-1775, Mubritinib, Mocetinostat, Pf-3758309, R428, Raltitrexed, XL228, Azd3514, Elinogrel, Tak-901, Chir-265, Bafetinib, Ac-480, Cc-401, Nilotinib, Radotinib, Golvatinib, Abexinostat, Ast-487, Enmd-2076, Bms-833923, Draflazine, Tezosentan, Lometrexol, Cinuperone, Icopezil, Elbanizine, Glisindamide, Camicinal, Tegobuvir, Flumeridone, Lomitapide, Mazokalim, Losulazine, Peraquinsin, Leflunomide, Afacifenacin, Ag-13958, Lorpiprazole, Siponimod, Mespiperone, XL019, Bentipimine, Zosuquidar, Fasitibant, Amg-517, Ci-988, Boxidine, Glicaramide, Niaprazine, Isometamidium, Metofenazate, Trenizine, Flotrenizine, Difluanine, Ramatroban, Sonepiprazole, Tafamidis, Verlukast, Ropizine, Lotrifen, Mioflazine, Talotrexin, Tiflamizole, Trefentanil, Asapiprant, Lidoflazine, Ruzadolane, Preladenant, Medibazine, Delavirdine, Piflutixol, Eberconazole, Teneligliptin, Daltroban, Deracoxib, Relenopride, Avagacestat, Dropropizine, Levodropropizine, MK2-AP, Nafamostat, and pharmaceutically acceptable salts thereof.

In certain embodiments, the MK2 activator is selected from the group consisting of Sepimostat, Bifepramide, Capeserod, Bifeprofen, Lorcinadol, Sulfamazone, Altapizone, Zaldaride, Netupitant, Casopitant, Pipamazine, Saperconazole, Barmastine, Bgt-226, Imidocarb, Elopiprazole, Oxaflumazine, Flupentixol, Clocapramine, Paliperidone, Tiospirone, Sertindole, Perospirone, Ziprasidone, Picloxydine, Amg-548, Cep-32496, Gsk-1070916, Gsk-461364, Ly-2584702, Fludoxopone, Clodoxopone, Amg-900, Mk-6592, Mk-8033, Azd-1775, Mubritinib, Mocetinostat, Pf-3758309, R428, Raltitrexed, XL228, Azd3514, Elinogrel, Tak-901, Chir-265, Bafetinib, Ac-480, Cc-401, Nilotinib, Radotinib, Golvatinib, Abexinostat, Ast-487, Enmd-2076, Bms-833923, Draflazine, Tezosentan, Lometrexol, Cinuperone, Icopezil-maleate, Elbanizine, Glisindamide, Camicinal, Tegobuvir, Flumeridone, Lomitapide, Mazokalim, Losulazine, Peraquinsin, Leflunomide, Afacifenacin, Ag-13958, Lorpiprazole, Siponimod, Mespiperone, XL019, Bentipimine, Zosuquidar, Fasitibant, Amg-517, Ci-988, Boxidine, Glicaramide, Niaprazine, Isometamidium, Metofenazate, Trenizine, Flotrenizine, Difluanine, Ramatroban, Sonepiprazole, Tafamidis, Verlukast, Ropizine, Lotrifen, Mioflazine, Talotrexin, Tiflamizole, Trefentanil, Asapiprant, Lidoflazine, Ruzadolane, Preladenant, Medibazine, Delavirdine, Piflutixol, Eberconazole, Teneligliptin, Daltroban, Deracoxib, Relenopride, Avagacestat, Dropropizine, Levodropropizine, MK2-AP, Nafamostat Mesylate, and combinations thereof.

In certain embodiments, the MK2 activator is selected from the group consisting of Sepimostat, Bifepramide, Capeserod, Bifeprofen, Lorcinadol, Sulfamazone, Altapizone, Zaldaride, Netupitant, Casopitant, Pipamazine, Saperconazole, Barmastine, Bgt-226, Imidocarb, Elopiprazole, Oxaflumazine, Flupentixol, Clocapramine, Paliperidone, Tiospirone, Sertindole, Perospirone, Ziprasidone, Picloxydine, Amg-548, Cep-32496, Gsk-1070916, Gsk-461364, Ly-2584702, Fludoxopone, Clodoxopone, Amg-900, Mk-6592, Mk-8033, Azd-1775, Mubritinib, Mocetinostat, Pf-3758309, R428, Raltitrexed, XL228, Azd3514, Elinogrel, Tak-901, Chir-265, Bafetinib, Ac-480, Cc-401, Nilotinib, Radotinib, Golvatinib, Abexinostat, Ast-487, Enmd-2076, Bms-833923, Draflazine, Tezosentan, Lometrexol, Cinuperone, Icopezil, Elbanizine, Glisindamide, Camicinal, Tegobuvir, Flumeridone, Lomitapide, Mazokalim, Losulazine, Peraquinsin, Leflunomide, Afacifenacin, Ag-13958, Lorpiprazole, Siponimod, Mespiperone, XL019, Bentipimine, Zosuquidar, Fasitibant, Amg-517, Ci-988, Boxidine, Glicaramide, Niaprazine, Isometamidium, Metofenazate, Trenizine, Flotrenizine, Difluanine, Ramatroban, Sonepiprazole, Tafamidis, Verlukast, Ropizine, Lotrifen, Mioflazine, Talotrexin, Tiflamizole, Trefentanil, Asapiprant, Lidoflazine, Ruzadolane, Preladenant, Medibazine, Delavirdine, Piflutixol, Eberconazole, Teneligliptin, Daltroban, Deracoxib, Relenopride, Avagacestat, and pharmaceutically acceptable salts thereof.

In certain embodiments, the MK2 activator is selected from the group consisting of Sepimostat, Bifepramide, Capeserod, Bifeprofen, Lorcinadol, Sulfamazone, Altapizone, Zaldaride, Netupitant, Casopitant, Pipamazine, Saperconazole, Barmastine, Bgt-226, Imidocarb, Elopiprazole, Oxaflumazine, Flupentixol, Clocapramine, Paliperidone, Tiospirone, Sertindole, Perospirone, Ziprasidone, Picloxydine, Amg-548, Cep-32496, Gsk-1070916, Gsk-461364, Ly-2584702, Fludoxopone, Clodoxopone, Amg-900, Mk-6592, Mk-8033, Azd-1775, Mubritinib, Mocetinostat, Pf-3758309, R428, Raltitrexed, XL228, Azd3514, Elinogrel, Tak-901, Chir-265, Bafetinib, Ac-480, Cc-401, Nilotinib, Radotinib, Golvatinib, Abexinostat, Ast-487, Enmd-2076, Bms-833923, Draflazine, Tezosentan, Lometrexol, Cinuperone, Icopezil-maleate, Elbanizine, Glisindamide, Camicinal, Tegobuvir, Flumeridone, Lomitapide, Mazokalim, Losulazine, Peraquinsin, Leflunomide, Afacifenacin, Ag-13958, Lorpiprazole, Siponimod, Mespiperone, XL019, Bentipimine, Zosuquidar, Fasitibant, Amg-517, Ci-988, Boxidine, Glicaramide, Niaprazine, Isometamidium, Metofenazate, Trenizine, Flotrenizine, Difluanine, Ramatroban, Sonepiprazole, Tafamidis, Verlukast, Ropizine, Lotrifen, Mioflazine, Talotrexin, Tiflamizole, Trefentanil, Asapiprant, Lidoflazine, Ruzadolane, Preladenant, Medibazine, Delavirdine, Piflutixol, Eberconazole, Teneligliptin, Daltroban, Deracoxib, Relenopride, Avagacestat, and combinations thereof.

In certain embodiments, the MK2 activator is selected from the group consisting of Sepimostat, Bifepramide, Capeserod, Bifeprofen, Lorcinadol, Sulfamazone, Altapizone, Zaldaride, Netupitant, Casopitant, Pipamazine, Saperconazole, Barmastine, Bgt-226, Imidocarb, Elopiprazole, Oxaflumazine, Flupentixol, Clocapramine, Paliperidone, Tiospirone, Sertindole, Perospirone, Ziprasidone, Picloxydine, Ly-2584702, Fludoxopone, Clodoxopone, Mubritinib, Mocetinostat, Pf-3758309, Raltitrexed, Azd3514, Elinogrel, Ac-480, Abexinostat, Bms-833923, Draflazine, Tezosentan, Lometrexol, Cinuperone, Icopezil-maleate, Elbanizine, Glisindamide, Camicinal, Tegobuvir, Flumeridone, Lomitapide, Mazokalim, Losulazine, Peraquinsin, Afacifenacin, Ag-13958, Lorpiprazole, Siponimod, Mespiperone, Bentipimine, Zosuquidar, Fasitibant, Amg-517, Ci-988, Boxidine, Glicaramide, Niaprazine, Isometamidium, Metofenazate, Flotrenizine, Difluanine, Ramatroban, Sonepiprazole, Tafamidis, Verlukast, Ropizine, Lotrifen, Mioflazine, Talotrexin, Tiflamizole, Trefentanil, Asapiprant, Lidoflazine, Ruzadolane, Preladenant, Medibazine, Delavirdine, Piflutixol, Eberconazole, Teneligliptin, Daltroban, Deracoxib, Relenopride, Avagacestat, and pharmaceutically acceptable salts thereof.

In certain embodiments, the MK2 activator is selected from the group consisting of Sepimostat, Bifepramide, Capeserod, Bifeprofen, Lorcinadol, Sulfamazone, Altapizone, Zaldaride, Netupitant, Casopitant, Pipamazine, Saperconazole, Barmastine, Bgt-226, Imidocarb, Elopiprazole, Oxaflumazine, Flupentixol, Clocapramine, Paliperidone, Tiospirone, Sertindole, Perospirone, Ziprasidone, Picloxydine, Ly-2584702, Fludoxopone, Clodoxopone, Mubritinib, Mocetinostat, Pf-3758309, Raltitrexed, Azd3514, Elinogrel, Ac-480, Abexinostat, Bms-833923, Draflazine, Tezosentan, Lometrexol, Cinuperone, Icopezil-maleate, Elbanizine, Glisindamide, Camicinal, Tegobuvir, Flumeridone, Lomitapide, Mazokalim, Losulazine, Peraquinsin, Afacifenacin, Ag-13958, Lorpiprazole, Siponimod, Mespiperone, Bentipimine, Zosuquidar, Fasitibant, Amg-517, Ci-988, Boxidine, Glicaramide, Niaprazine, Isometamidium, Metofenazate, Flotrenizine, Difluanine, Ramatroban, Sonepiprazole, Tafamidis, Verlukast, Ropizine, Lotrifen, Mioflazine, Talotrexin, Tiflamizole, Trefentanil, Asapiprant, Lidoflazine, Ruzadolane, Preladenant, Medibazine, Delavirdine, Piflutixol, Eberconazole, Teneligliptin, Daltroban, Deracoxib, Relenopride, Avagacestat, and combinations thereof.

In certain embodiments, the MK2 activator is selected from the group consisting of Abexinostat (PCI-24781), Ac-480 (BMS-599626), Adavosertib (MK-1775), Ag-13958, Amg-517, Amg-900, Asapiprant, Ast-487 (NVP-AST487), Avagacestat (BMS-708163), Azd3514, BAF312 (Siponimod), Bafetinib (INNO-406), Bemcentinib (R428), Bgt-226 (NVP-BGT226), Bms-833923, Cc-401, Cep-32496, Delavirdine, Deracoxib, Dropropizine, Enmd-2076, Flupentixol, Golvatinib, Gsk-1070916, Gsk-461364, Imidocarb, Leflunomide, Levodropropizine, Lomitapide, Ly-2584702, MK2-AP, Mocetinostat (MGCD0103), Mubritinib (TAK 165), Nafamostat Mesylate, Netupitant, Nilotinib (AMN-107), Paliperidone, Perospirone, Pf-3758309, Preladenant, Radotinib, RAF265 (Chir-265), Raltitrexed, Ramatroban, Tafamidis, Tak-901, Tegobuvir, Teneligliptin, XL019, XL228, Ziprasidone, Zosuquidar (LY335979), and pharmaceutically acceptable salts thereof.

In certain embodiments, the MK2 activator is selected from the group consisting of Abexinostat (PCI-24781), Ac-480 (BMS-599626), Adavosertib (MK-1775), Ag-13958, Amg-517, Amg-900, Asapiprant, Ast-487 (NVP-AST487), Avagacestat (BMS-708163), Azd3514, BAF312 (Siponimod), Bafetinib (INNO-406), Bemcentinib (R428), Bgt-226 (NVP-BGT226), Bms-833923, Cc-401 Hydrochloride, Cep-32496, Delavirdine (mesylate), Deracoxib, Dropropizine, Enmd-2076, Flupentixol dihydrochloride, Golvatinib, Gsk-1070916, Gsk-461364, Imidocarb dipropionate, Leflunomide, Levodropropizine, Lomitapide, Ly-2584702, MK2-AP (batch #2), Mocetinostat (MGCD0103), Mubritinib (TAK 165), Nafamostat Mesylate, Netupitant, Nilotinib (AMN-107), Paliperidone, Perospirone hydrochloride, Pf-3758309, Preladenant, Radotinib, RAF265 (Chir-265), Raltitrexed, Ramatroban, Tafamidis, Tak-901, Tegobuvir, Teneligliptin hydrobromide, XL019, XL228, Ziprasidone HCl, Zosuquidar (LY335979) 3HCl, and combinations thereof.

In certain embodiments, the MK2 activator is selected from the group consisting of Lomitapide, Perospirone, Abexinostat, AG-13958, Levodropropizine, Nilotinib, AMG-900, AMG-517, Delavirdin, Bgt-226, Mocetinostat, Tafamidis, XL019, Radotinib, Ziprasidone, Mubritinib, Ramatroban, Deracoxib, Raltitrexed, GSK461364, Adavosertib, Tegobuvir, and pharmaceutically acceptable salts thereof.

In certain embodiments, the MK2 activator is selected from the group consisting of Lomitapide, Perospirone, Abexinostat, AG-13958, Levodropropizine, Nilotinib, AMG-900, AMG-517, Delavirdin, Bgt-226, Mocetinostat, Tafamidis, XL019, Radotinib, Ziprasidone, Mubritinib, Ramatroban, Deracoxib, Raltitrexed, GSK461364, Adavosertib, Tegobuvir, and combinations thereof.

In certain embodiments, the MK2 activator is selected from the group consisting of Leflunomide, Netupitant, Paliperidone, Lomitapide, Perospirone, and pharmaceutically acceptable salts thereof.

In certain embodiments, the MK2 activator is selected from the group consisting of Leflunomide, Netupitant, Paliperidone, Lomitapide, Perospirone hydrochloride, and combinations thereof.

TABLE 2
List of MK2 Activator Compounds

| Compound Number | Compound Name |
|---|---|
| 1 | Sepimostat |
| 2 | Bifepramide |
| 3 | Capeserod |
| 4 | Bifeprofen |
| 5 | Lorcinadol |
| 6 | Sulfamazone |
| 7 | Altapizone |
| 8 | Zaldaride |
| 9 | Netupitant |
| 10 | Casopitant |
| 11 | Pipamazine |
| 12 | Saperconazole |
| 13 | Barmastine |
| 14 | Bgt-226 |
| 15 | Imidocarb |
| 16 | Elopiprazole |
| 17 | Oxaflumazine |
| 18 | Flupentixol |
| 19 | Clocapramine |
| 20 | Paliperidone |
| 21 | Tiospirone |
| 22 | Sertindole |
| 23 | Perospirone |
| 24 | Ziprasidone |
| 25 | Picloxydine |
| 26 | Amg-548 |
| 27 | Cep-32496 |
| 28 | Gsk-1070916 |
| 29 | Gsk-461364 |
| 30 | Ly-2584702 |
| 31 | Fludoxopone |
| 32 | Clodoxopone |
| 33 | Amg- 900 |
| 34 | Mk-6592 |
| 35 | Mk- 8033 |
| 36 | Azd-1775 |
| 37 | Mubritinib |
| 38 | Mocetinostat |
| 39 | Pf-03758309 |
| 40 | R428 |
| 41 | Raltitrexed |
| 43 | Xl-228 |
| 44 | Azd3514 |
| 45 | Elinogrel |
| 46 | Tak-901 |
| 47 | Chir-265 |
| 48 | Bafetinib |
| 49 | Ac-480 |
| 50 | Cc-401 |
| 51 | Nilotinib |
| 52 | Radotinib |
| 53 | Golvatinib |
| 54 | Abexinostat |
| 55 | Ast-487 |
| 56 | Enmd-2076 |
| 57 | Bms - 833923 |
| 58 | Draflazine |
| 59 | Tezosentan |
| 60 | Lometrexol |
| 61 | Cinuperone |
| 62 | Icopezil-maleate |
| 63 | Elbanizine |
| 64 | Glisindamide |
| 65 | Camicinal |
| 66 | Tegobuvir |
| 67 | Flumeridone |
| 68 | Lomitapide |
| 69 | Mazokalim |
| 70 | Losulazine |
| 71 | Peraquinsin |
| 72 | Leflunomide |
| 73 | Afacifenacin |
| 74 | Ag-13958 |
| 75 | Lorpiprazole |
| 76 | Siponimod |
| 77 | Mespiperone |
| 78 | Xl-019 |
| 80 | Bentipimine |
| 81 | Zosuquidar |
| 82 | Fasitibant |
| 83 | Amg-517 |
| 84 | Ci-988 |
| 85 | Boxidine |
| 86 | Glicaramide |
| 87 | Niaprazine |
| 88 | Isometamidium |
| 89 | Metofenazate |
| 90 | Trenizine |
| 91 | Flotrenizine |
| 92 | Difluanine |
| 93 | Ramatroban |
| 94 | Sonepiprazole |
| 95 | Tafamidis |
| 96 | Verlukast |
| 97 | Ropizine |
| 98 | Lotrifen |
| 99 | Mioflazine |
| 100 | Talotrexin |
| 101 | Tiflamizole |
| 102 | Trefentanil |
| 103 | Asapiprant |
| 104 | Lidoflazine |
| 105 | Ruzadolane |
| 106 | Preladenant |
| 107 | Medibazine |
| 108 | Delavirdine |
| 109 | Piflutixol |
| 110 | Eberconazole |
| 111 | Teneligliptin |
| 112 | Daltroban |
| 113 | Deracoxib |
| 114 | Relenopride |
| 115 | Avagacestat |

In another aspect, provided herein are compositions comprising an MK2 activator, or a pharmaceutically acceptable salt thereof. In certain embodiments, the MK2 activator is an MK2 activator described herein, or a pharmaceutically acceptable salt thereof (e.g., an MK2 activator selected from any compound set forth in Table 2).

In various embodiments, the compositions described herein can be useful for treatment, prophylaxis and reduction in the risk of a vascular disorder or an endothelium barrier disorder.

The compositions of this disclosure can be administered in an amount sufficient to improve one or more symptoms of a disorder described herein (e.g., a vascular disorder, an endothelium barrier disorder) in a patient in need thereof.

In various embodiments, provided herein are compositions for the treatment of a vascular disorder or an endothelial barrier disorder comprising a therapeutically effective amount of an MK2 activator described herein.

Furthermore, the compositions provided herein can be administered by a variety of routes including, but not limited to, oral (enteral) administration, parenteral (by injection) administration, rectal administration, transdermal administration, intradermal administration, intrathecal administration, subcutaneous (SC) administration, intravenous (IV) administration, intramuscular (I) administration, inhalation, nebulization, and intranasal administration.

In certain embodiments, the composition is administered intravenously, orally, subcutaneously, or intramuscularly. In certain embodiments, the composition is administered intravenously. In certain embodiments, the composition is administered orally. In certain embodiments, the composition is administered parenterally. In some embodiments, the composition is administered chronically. In some embodiments, the composition is administered continuously (e.g., by continuous intravenous infusion).

Furthermore, a composition provided herein can be presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In another aspect, provided herein are unit dosage forms for the treatment of a vascular disorder or an endothelial barrier disorder comprising a therapeutically effective amount of an MK2 activator described herein.

### Pharmaceutical Compositions

In one aspect, provided herein are pharmaceutical compositions comprising an MK2 activator, or a pharmaceutically acceptable salt thereof, and a pharmaceutically acceptable excipient. In certain embodiments, the MK2 activator is an MK2 activator described herein, or a pharmaceutically acceptable salt thereof (e.g., an MK2 activator selected from any compound set forth in Table 2).

In various embodiments, the pharmaceutical compositions described herein can be useful for treatment, prophylaxis and reduction in the risk of a vascular disorder or an endothelium barrier disorder.

The pharmaceutical compositions of this disclosure can be administered in an amount sufficient to improve one or more symptoms of a disorder described herein (e.g., a vascular disorder, an endothelium barrier disorder) in a patient in need thereof.

In certain embodiments, the MK2 activator of the present disclosure is provided in an effective amount in the pharmaceutical composition. In certain embodiments, the MK2 activator of the present disclosure is provided in a therapeutically effective amount.

In various embodiments, provided herein are pharmaceutical compositions for the treatment of a vascular disorder or an endothelial barrier disorder comprising a therapeutically effective amount of an MK2 activator described herein.

The pharmaceutical compositions provided herein can be administered by a variety of routes including, but not limited to, oral (enteral) administration, parenteral (by injection) administration, rectal administration, transdermal administration, intradermal administration, intrathecal administration, subcutaneous (SC) administration, intravenous (IV) administration, intramuscular (I) administration, inhalation, nebulization, and intranasal administration.

In another aspect, the disclosure provides a pharmaceutical composition comprising a composition of the present disclosure and a pharmaceutically acceptable excipient, e.g., a composition suitable for injection, such as for intravenous (IV) administration.

In certain embodiments, the pharmaceutical composition is administered intravenously, orally, subcutaneously, or intramuscularly. In certain embodiments, the pharmaceutical composition is administered intravenously. In certain embodiments, the pharmaceutical composition is administered orally. In certain embodiments, the pharmaceutical composition is administered parenterally. In some embodiments, the pharmaceutical composition is administered chronically. In some embodiments, the pharmaceutical composition is administered continuously (e.g., by continuous intravenous infusion).

Furthermore, a pharmaceutical composition described herein can be presented in unit dosage forms to facilitate accurate dosing.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation. General considerations in the formulation and/or manufacture of pharmaceutical compositions can be found, for example, in Remington: The Science and Practice of Pharmacy 21^{st} ed., Lippincott Williams & Wilkins, 2005.

### Methods of Use and Treatment

The MK2 activators described herein can be useful in treating patients suffering from a disorder described herein. For example, the methods of the present disclosure can be useful for the activation of phosphorylation of the small heat shock protein HSP-27 to augment the endothelial barrier. Accordingly, the methods of the present disclosure can be useful for treating patients suffering from, for example, a vascular disorder or an endothelial barrier disorder (e.g., acute respiratory distress syndrome). Examples of the disorders that can be treated are described below.

In various embodiments, provided herein are methods of treating a patient suffering from a disorder described herein, comprising administering to the patient a therapeutically effective amount of an MK2 activator described herein (e.g., an MK2 activator selected from any compound set forth in Table 2).

An aspect of the disclosure provides methods of treating patients suffering from a vascular disorder, e.g., vasculitis, vascular leak disorder, abdominal aortic aneurysm, atherosclerosis, carotid artery disease/carotid artery stenosis, chronic venous insufficiency, intermittent claudication, deep vein thrombosis, peripheral vascular disease, pulmonary embolism, pulmonary hypertension, cerebral ischemia, stroke, Raynaud's phenomenon, renal vascular disease, thoracic aortic aneurysm, heart attack, and varicose veins. In particular, in certain embodiments, the disclosure provides a method of treating the below medical indications comprising administering to a patient in need thereof a therapeutically effective amount of an MK2 activator.

In certain embodiments, the vascular disorder is a vascular leak disorder.

In various embodiments, the vascular disorder is associated with the endothelium. In certain embodiments, the vascular disorder is an endothelium barrier disorder. In certain embodiments, the endothelial barrier disorder is a microvascular barrier leak disorder.

In certain embodiments, the vascular disorder is a vasculitis. In some embodiments, the vasculitis is selected from the group consisting of large vessel vasculitis, medium vessel vasculitis, and small vessel vasculitis. In some embodiments, the large vessel vasculitis is selected from takayasu's arteritis and temporal arteritis. In some embodiments, the medium vessel vasculitis is selected from the group consisting of Buerger's disease, Kawasaki disease, and Polyarteritis nodosa. In some embodiments, the small vessel vasculitis is selected from the group consisting of Behçet's syndrome, Eosinophilic granulomatosis with polyangiitis (also known as Churg-Strauss syndrome), Cutaneous vasculitis, granulomatosis with polyangiitis, Henoch-Schonlein purpura, and microscopic polyangiitis. In certain embodiments, the eosinophilic granulomatosis with polyangiitis (also known as Churg-Strauss syndrome) is associated with acute respiratory distress syndrome. In certain embodiments, the granulomatosis with polyangiitis is associated with pneumonia.

In some embodiments, the vascular disorder is associated with (e.g., induced by) a virus. In certain embodiments, the vascular disorder is associated with (e.g., induced by) a virus selected from the group consisting of an RNA virus, a DNA virus, a coronavirus, a papillomavirus, a pneumovirus, an influenza virus, an adeno-associated virus, an adenovirus, a baculovirus, a retrovirus, a lentivirus, an alphavirus, a bunyavirus (e.g., hantavirus), a filovirus (e.g., Ebola virus), and an arbovirus (e.g., yellow fever virus, Dengue virus). In certain embodiments, the vascular disorder is associated with (e.g., induced by) a coronavirus. In some embodiments, the coronavirus associated with the vascular disorder is selected from the group consisting of 229E alpha coronavirus, NL63 alpha coronavirus, OC43 beta coronavirus, HKU1 beta coronavirus, Middle East Respiratory Syndrome coronavirus (MERS-CoV), severe acute respiratory syndrome coronavirus (SARS-CoV), and coronavirus diseases 2019 (SARS-CoV-2 or COVID-19). In some embodiments, the coronavirus associated with the vascular disorder is COVID-19.

In other embodiments, the virus is selected from the group consisting of canarypox virus, infectious bovine rhinotracheitis virus, bovine viral diarrhea virus, parainfluenza 3 virus, bovine respiratory syncytial virus, feline calicivirus, chlamydia virus, canine coronavirus, panleukopenia virus, feline leukemia virus, hepatitis A, hepatitis B, and hepatitis C, human immunodeficiency virus type 1 (HIV-1) and type 2 (HIV-2), cytomegalovirus, human T-lymphotropic virus type I (HTLV-I) and type II (HTLV-II), encephalitis virus, measles virus, mumps virus, rubella virus, polio virus, rabies virus, respiratory syncytial virus, rotavirus, smallpox virus, typhoid vaccine virus, varicella virus, and yellow fever vaccine virus.

In embodiments, the vascular disorder is associated with (e.g., induced by) a bacteria. In certain embodiments, the bacteria is selected from the group consisting of Streptococcus pneumoniae, Staphylococcus aureus, Group A Streptococcus, Klebsiella pneumoniae, Haemophilus influenzae, Moraxella catarrhalis, anaerobes, Bacillus anthracis (anthrax), and gram-negative organisms.

Another aspect of the disclosure provides methods of treating patients suffering from an endothelial barrier disorder, e.g., microvascular barrier leak disorder, blood vessels barrier leak disorder, vascular barrier disorder, and gastrointestinal disorder. In certain embodiments, the microvascular barrier leak disorder is selected from the group consisting of inflammatory bowel disease, brain edema, acute lung injury, acute respiratory distress syndrome, and pulmonary edema. In some embodiments, the blood vessel barrier leak disorder is selected from the group consisting of atherosclerosis, hypertension, preeclampsia, and Kawasaki disease. In some embodiments, the endothelial barrier disorder is a vascular barrier disorder. In some embodiments, the vascular barrier disorder is a fibroblasts-related disorder. In some embodiments, the fibroblasts-related disorder is selected from the group consisting of wound healing, pulmonary fibrosis, pulmonary fibrosis, liver fibrosis, vascular fibrosis, kidney fibrosis, and tissue remodeling. In some embodiments, the gastrointestinal disorder is selected from the group consisting of celiac disease, constipation, Crohn's disease, diarrhea, diverticular disease, Gastroesophageal Reflux Disease (GERD), hemorrhoids and anal fissures, irritable bowel syndrome, lactose intolerance, malabsorption syndromes, polyps and colorectal cancer, Peptic Ulcer Disease (PUD), ulcerative colitis, and vomiting. In particular, in certain embodiments, the disclosure provides a method of treating the below medical indications comprising administering to a patient in need thereof a therapeutically effective amount of an MK2 activator.

In various embodiments, the endothelial barrier disorder is associated with a hyper-permeability of an endothelial barrier. In some embodiments, the hyper-permeability of an endothelial barrier is associated with (e.g., induced by) COVID-19. In some embodiments, the hyper-permeability of an endothelial barrier is associated with acute respiratory distress syndrome. In some embodiments, the hyper-permeability endothelial barrier is associated with acute lung injury.

In some embodiments, the endothelial barrier disorder is associated with (e.g., induced by) a virus. In certain embodiments, the hyper-permeability endothelial barrier is associated (e.g., induced) with a virus. For example, the virus is selected from the group consisting of an RNA virus, a DNA virus, a coronavirus, a papillomavirus, a pneumovirus, an influenza virus, an adeno-associated virus, an adenovirus, a baculovirus, a retrovirus, a lentivirus, an alphavirus, a bunyavirus (e.g., hantavirus), a filovirus (e.g., Ebola virus), and an arbovirus (e.g., yellow fever virus, Dengue virus). In certain embodiments, the endothelial barrier disorder is associated with a coronavirus. In some embodiments, the coronavirus associated with the endothelial barrier disorder is selected from the group consisting of 229E alpha coronavirus, NL63 alpha coronavirus, OC43 beta coronavirus, HKU1 beta coronavirus, Middle East Respiratory Syndrome coronavirus (MERS-CoV), severe acute respiratory syndrome coronavirus (SARS-CoV), and coronavirus diseases 2019 (SARS-CoV-2 or COVID-19). In some embodiments, the coronavirus associated with the endothelial barrier disorder is COVID-19.

In other embodiments, the virus is selected from the group consisting of canarypox virus, infectious bovine rhinotracheitis virus, bovine viral diarrhea virus, parainfluenza 3 virus, bovine respiratory syncytial virus, feline calicivirus, chlamydia virus, canine coronavirus, panleukopenia virus, feline leukemia virus, hepatitis A, hepatitis B, and hepatitis C, human immunodeficiency virus type 1 (HIV-1) and type 2 (HIV-2), cytomegalovirus, human T-lymphotropic virus type I (HTLV-I) and type II (HTLV-II), encephalitis virus, measles virus, mumps virus, rubella virus, polio virus, rabies virus, respiratory syncytial virus, rotavirus, smallpox virus, typhoid vaccine virus, varicella virus, and yellow fever vaccine virus.

In embodiments, the endothelial barrier disorder is associated with (e.g., induced by) a bacteria. In certain embodiments, the hyper-permeability endothelial barrier is associated with (e.g., induced by) a bacteria. In certain embodiments, the bacteria is selected from the group consisting of Streptococcus pneumoniae, Staphylococcus aureus, Group A Streptococcus, Klebsiella pneumoniae, Haemophilus influenzae, Moraxella catarrhalis, anaerobes, Bacillus anthracis (anthrax), and gram-negative organisms.

In various embodiments, provided herein are methods of treating a patient suffering from acute respiratory distress syndrome. In certain embodiments, provided herein are methods of treating a patient suffering from acute respiratory distress syndrome, comprising administering to the patient a therapeutically effective amount of an MK2 activator. In certain embodiments, the acute respiratory distress syndrome is associated with (e.g., induced by) with a virus. In some embodiments, the acute respiratory distress syndrome is associated with (e.g., induced by) a virus selected from the group consisting of an RNA virus, a DNA virus, a coronavirus, a papillomavirus, a pneumovirus, an influenza virus, an adeno-associated virus, an adenovirus, a baculovirus, a retrovirus, a lentivirus, and an alphavirus. In some embodiments, the coronavirus associated with the acute respiratory distress syndrome is selected from the group consisting of 229E alpha coronavirus, NL63 alpha coronavirus, OC43 beta coronavirus, HKU1 beta coronavirus, Middle East Respiratory Syndrome coronavirus (MERS-CoV), severe acute respiratory syndrome coronavirus (SARS-CoV), and coronavirus diseases 2019 (SARS-CoV-2 or COVID-19). In certain embodiments, the acute respiratory distress syndrome is associated with (e.g., induced by) COVID-19.

In various embodiments, provided herein are methods of treating a patient suffering from an acute lung injury. In certain embodiments, provided herein are methods of treating a patient suffering from an acute lung injury, comprising administering to the patient a therapeutically effective amount of an MK2 activator. In certain embodiments, the acute lung injury is associated with (e.g., induced by) a virus. In some embodiments, the acute lung injury is associated with (e.g., induced by) a virus selected from the group consisting of an RNA virus, a DNA virus, a coronavirus, a papillomavirus, a pneumovirus, an influenza virus, an adeno-associated virus, an adenovirus, a baculovirus, a retrovirus, a lentivirus, and an alphavirus. In some embodiments, the coronavirus associated with the acute lung injury is selected from the group consisting of 229E alpha coronavirus, NL63 alpha coronavirus, OC43 beta coronavirus, HKU1 beta coronavirus, Middle East Respiratory Syndrome coronavirus (MERS-CoV), severe acute respiratory syndrome coronavirus (SARS-CoV), and coronavirus diseases 2019 (SARS-CoV-2 or COVID-19). In certain embodiments, the acute lung injury is associated with (e.g., induced by) COVID-19.

In certain embodiments, the method comprises administering compositions as described herein to the patient an amount sufficient to improve one or more symptoms of a vascular disorder or an endothelial barrier disorder.

In certain embodiments, the MK2 activator is administered intravenously to the patient. In certain embodiments, the MK2 activator is administered orally to the patient. In certain embodiments, the MK2 activator is administered parenterally to the patient.

In certain embodiments, the patient is a human.

### Definitions

To facilitate an understanding of the present invention, a number of terms and phrases are defined below.

Unless defined otherwise, all technical and scientific terms used herein have the same meaning as commonly understood by one of ordinary skill in the art to which this invention belongs. The abbreviations used herein have their conventional meaning within the chemical and biological arts. The chemical structures and formulae set forth herein are constructed according to the standard rules of chemical valency known in the chemical arts.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The terms “a” and “an” as used herein mean “one or more” and include the plural unless the context is inappropriate.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred from the context.

As used herein, the terms “disorder,” “disease,” and “condition” are used interchangeably.

As used herein, the term “effective amount” refers to the amount of a composition (e.g., a liquid pharmaceutical formulation of the present invention) sufficient to effect beneficial or desired results. An effective amount can be administered in one or more administrations, applications or dosages and is not intended to be limited to a particular formulation or administration route.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the formulation and are biologically acceptable. In certain embodiments, supplementary active ingredients can also be incorporated into the pharmaceutical compositions.

As used herein, “pharmaceutically acceptable excipient” refers to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, a phosphate buffered saline solution, emulsions (e.g., such as an oil/water or water/oil emulsions), lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxypropylmethylcellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. For examples of excipients and carriers, see Martin, Remington's Pharmaceutical Sciences, 15th Ed., Mack Publ. Co., Easton, PA (1975).

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see Martin, Remington's Pharmaceutical Sciences, 15th Ed., Mack Publ. Co., Easton, PA [1975].

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

As used herein, the terms “subject” and “patient” refer to organisms to be treated by the methods and/or compositions described herein. Such organisms are preferably mammals (e.g., murines, simians, equines, bovines, porcines, canines, felines, and the like), and more preferably humans.

As used herein, the terms “treat,” “treating,” and “treatment” include any effect, e.g., lessening, reducing, modulating, ameliorating or eliminating, that results in the improvement of the condition, disease, disorder, and the like, or ameliorating a symptom thereof.

The phrase “therapeutically-effective amount” as used herein means that amount of a composition (e.g., a liquid pharmaceutical formulation of the present invention) which is effective for producing some desired therapeutic effect in a subject.

### EXAMPLES

In order that the disclosure described herein may be more fully understood, the following examples are set forth. The examples described in this application are offered to illustrate the compounds, pharmaceutical compositions, and methods provided herein and are not to be construed in any way as limiting their scope.

### Example 1: MK2 Loop Construction Methods

Three missing loops—residues 153-158 (RGDQAF (SEQ ID NO: 4)), 215-238 (TSHNSLTTPCYTPYYVAPEVLGPE (SEQ ID NO: 5)), and 265-273 (SNHGLAISP (SEQ ID NO: 6))—in the crystal structure of MK2 Kinase co-crystallized with a small catalytic site inhibitor (PDB ID: 3KA0) (PMID 19919896) were constructed using the interactive Superlooper2 web-server (reference: pubmed id: PMID: 27105847).

Grafted loops were then initially energy minimized in vacuum with the steepest descent algorithm with the GROMACS v5.1.3 standalone package (ref: Abraham M. J., Murtola T., Schulz R., Pill S., Smith J. C., Hess B., Lindahl E. GROMACS: High performance molecular simulations through multi-level parallelism from laptops to supercomputers. SoftwareX. 2015; 1:19-25. doi: 10.1016/j.softx.2015.06.001.) while the remaining protein atoms remained fixed.

### Example 2: MK2 Loop Refinement Methods

The initial model structures were then solvated (with the correct number of counterions added) and further equilibrated in GROMACS through a series of eight MD simulations (N, amount of substance, V, volume, T, temperature canonical ensemble), each one for a duration of 500 picoseconds and with different initial parameters (temperature, thermostat, and/or interaction cutoff radius) in order to obtain different starting points for the runs. Then NPT run (isothermal-isobaric ensemble, P for pressure) for each one of the loop structures were carried out followed by 4 nanosecond simulation using the same parameters. All the obtained structures were then evaluated regarding the system's final energies and a Ramachandran plot for structural stability. The lowest energy structures with a favorable Ramachandran plot for the three loops (RGDQAF (SEQ ID NO: 4), TSHNSLTTPCYTPYYVAPEVLGPE (SEQ ID NO: 5), and SNHGLAISP (SEQ ID NO: 6)) that required modeling in the present study were selected for the final model.

### Example 3: MK2-Activating Peptide Docking Methods

The CABS-dock standalone package (PMID: 31682301; PMID: 30865258) and the MDockPeP server were used to dock the positively charged activating peptide YARAAARQARAHPRNPARRTPGTRRGAPAA (SEQ ID NO: 7) (PMID: 26066827) onto the surface of MK2 model structure.

1. Peptide Docking with the CABS-Dock Standalone Application

The docking protocol consisted of four steps: First, the MK2 receptor structure was converted to the CABS coarse-grained representation (a single amino acid is represented by up to four atoms or pseudo-atoms). Next, random peptide structures were randomly selected from a library of generic peptide conformations (the same type of coarse-grained representations were also used for the peptides) and placed in random positions around the MK2 receptor, in the approximate distance of 20 Å from the receptor surface. The MK2 3D structure and the peptides are considered flexible during the simulation. Second, filtering of the docked peptides was based on the computed CABS MK2-peptide interaction energy values (1000 low-energy models were selected from 10000 conformations generated during the docking simulations). Third, clustering and scoring of the final models (100 top-scored models were selected from 1000 low-energy models). Fourth, reconstruction of the 10 final best MK2-peptide models to an all-atom representation was performed. The resulting MK2-peptide structures were visually inspected in Maestro and PyMol.

2. Peptide Docking with the MDockPeP Server

A binding docking experiment was carried out with the MDockPeP ab-initio protein-peptide docking server (PMID: 30368849). First, the MDockPeP server modeled up to 3 non-redundant conformers for our input peptide based on similar-sequence fragments from monomeric proteins. Next, the modeled peptide conformers were independently docked to the whole MK2 protein using a modified version of the AutoDockVina package (PMID: 19499576). The grid box for the search was defined by extending 20 Å to both the minimum and the maximum of the coordinates of the protein structure in three dimensions. The peptide conformers are then rigidly docked to the whole protein by randomly generating 105 translational and rotational configurations within the grid box. These preliminary models were initially ranked using the Vina scoring function. Then, flexible sampling was performed for the docked peptide models that have the best Vina docking scores. All rotatable bonds in the peptide were treated as flexible during sampling. At this stage, all these initial binding modes generated from different initial peptide conformers were combined and then re-ranked according to energy scores computed with ITScorePeP (PMID: 27642160), a statistical potential-based scoring function specifically developed for protein-peptide dockings. Contributions from both interactions between the protein and the peptide (inter-score) and interactions among non-neighbored residues within the peptide (intra-score) are considered in the scoring function. The top 100 binding modes were then analyzed interactively with the PyMol molecular graphics package.

### Example 4: MK2 Molecular Surface Analysis and Binding Pocket Prediction Methods

Electrostatic calculations were computed with the Adaptive Poisson-Boltzmann Solver (APBS) (PMID: 28836357) in order to search for putative strong electronegative binding grooves that could be critical for the interaction with the positively charged peptide. Prediction of likely binding pockets for small molecules was performed with the P2Rank (PMID: 30109435), a machine learning-based tool to propose putative ligand binding sites. The MK2 3D structure was used as input and the top 5 predicted pockets were investigated interactively in the light of electrostatic computations and the full surface peptide docking experiments mentioned above.

### Example 5: Drug Screening Assay with Structure-Based Computations

The MTiOpenScreen web-server with the Drugs-lib collection (PMID: 30190791 PMID: 25855812) was used and docking was performed on the most likely MK2 peptide binding pocket predicted. This server implements AutoDock Vina for structure-based virtual screening and scoring tool (PMID: 27077332). A list of about 22,000 approved and investigational drugs were obtained from DrugBank [PMID: 29126136], DrugCentral [PMID: 30371892], SuperDrug2 [PMID: 29140469], and ChEMBL [PMID: 30398643]. A list of the compounds can also be found in the Drugs-lib collection [PMID:30190791].

Molecules were curated, duplicates were removed and filtering was performed to reject compounds with strong documented toxicophores using FAF-Drug4 [PMID: 28961788], and molecules with less than 20 rotatable bonds and a MW below 1000 Da were identified for the study. From the Drugs-lib collection, molecules that are available in the chemical vendor catalogs were identified for the study which summed to about 4600 molecules. The protonation states for the compound titratable groups were predicted using ChemAxon (https://chemaxon.com/) and the 3D structures were built using Corina (https://www.mn-am.com/). The top 1500 best docking scores were then considered and analyzed interactively to guide the selection of molecules.

### Example 6: Identification of MK2-Activator Drug Candidates

Electrostatic computations highlighted a strong electronegative groove in the C-lobe domain of MK2, away from the catalytic site just below the autoinhibitory domain which have sequence identity to amino acids 345-390 (SEQ ID NO: 8). This region is essentially located between E290 and D351 and nearby the binding interface of p38a as seen in the crystal structure of the MK2-p38 complex (PMID 17395714). Binding pocket predictions was performed and two of the top 3 binding pockets (pockets 1 and 3) were predicted in the electronegative. Pocket 1 includes the amino acid residues M275, K276, I279, V352, E354, E355, S358, A359, and T362 of SEQ ID NO: 1. Pocket 3 includes the amino acid residues Y260, Y264, S265, N266, H267, E285, F286, P287, N288, P289, E290, and V341. The second ranked binding pocket corresponds to the catalytic site. Two full surface peptide docking algorithms were used to position the activating peptide on the MK2 surface and both methods predicted the most likely binding site to be in the electronegative groove and aligned with the predicted pockets 1 and 3. Taken together, these result suggested that the region of pockets 1 and 3 could be used for structure-based drug repurposing computations. Pockets 1 and 3 were merged into a single search zone and structure-based virtual screening was carried out over this region, and the residues of SED ID NO: 1 that are present in this overall pocket include Y260, Y264, S265, N266, H267, A270, I271, S272, P273, G274, M275, K276, R278, 1279, E285, F286, P287, N288, P289, E290, V341, E347, R348, E350, D351, V352, E354, E355, S358, A359, and T362. The top 1500 scores were analyzed interactively on the computer screen and the top 115 compounds that fitted well into the cavity and those with favorable docking scores were identified (Table 3). Some compounds with better scores were disregarded because their binding poses and interaction patterns with MK2 amino acids were found unlikely after 3D visualization in PyMol.

TABLE 3

| Compound | Compound | Docking Energy, |
|---|---|---|
| Number | Name | kcal/mol |
| 1 | Sepimostat | −8.1 |
| 2 | Bifepramide | −7.2 |
| 3 | Capeserod | −7.5 |
| 4 | Bifeprofen | −8.2 |
| 5 | Lorcinadol | −7.2 |
| 6 | Sulfamazone | −7.7 |
| 7 | Altapizone | −7.3 |
| 8 | Zaldaride | −7.4 |
| 9 | Netupitant | −8.2 |
| 10 | Casopitant | −7.8 |
| 11 | Pipamazine | −7.2 |
| 12 | Saperconazole | −7.8 |
| 13 | Barmastine | −7.7 |
| 14 | Bgt-226 | −7.7 |
| 15 | Imidocarb | −7.3 |
| 16 | Elopiprazole | −8.1 |
| 17 | Oxaflumazine | −7.3 |
| 18 | Flupentixol | −7.2 |
| 19 | Clocapramine | −7.7 |
| 20 | Paliperidone | −7.6 |
| 21 | Tiospirone | −7.2 |
| 22 | Sertindole | −7.2 |
| 23 | Perospirone | −7.2 |
| 24 | Ziprasidone | −7.8 |
| 25 | Picloxydine | −7.7 |
| 26 | Amg-548 | −7.6 |
| 27 | Cep-32496 | −7.7 |
| 28 | Gsk-1070916 | −7.4 |
| 29 | Gsk-461364 | −7.3 |
| 30 | Ly-2584702 | −7.5 |
| 31 | Fludoxopone | −7.5 |
| 32 | Clodoxopone | −7.0 |
| 33 | Amg- 900 | −8.1 |
| 34 | Mk-6592 | −7.4 |
| 35 | Mk- 8033 | −7.9 |
| 36 | Azd-1775 | −7.7 |
| 37 | Mubritinib | −8.5 |
| 38 | Mocetinostat | −8.4 |
| 39 | Pf-03758309 | −7.4 |
| 40 | R428 | −9.4 |
| 41 | Raltitrexed | −7.7 |
| 43 | Xl-228 | −7.9 |
| 44 | Azd3514 | −8.0 |
| 45 | Elinogrel | −7.7 |
| 46 | Tak-901 | −7.7 |
| 47 | Chir-265 | −8.5 |
| 48 | Bafetinib | −8.0 |
| 49 | Ac-480 | −7.7 |
| 50 | Cc-401 | −7.6 |
| 51 | Nilotinib | −8.5 |
| 52 | Radotinib | −8.4 |
| 53 | Golvatinib | −9.1 |
| 54 | Abexinostat | −7.0 |
| 55 | Ast-487 | −7.6 |
| 56 | Enmd-2076 | −7.1 |
| 57 | Bms - 833923 | −8.3 |
| 58 | Draflazine | −7.6 |
| 59 | Tezosentan | −7.5 |
| 60 | Lometrexol | −8.1 |
| 61 | Cinuperone | −7.0 |
| 62 | Icopezil-maleate | −7.7 |
| 63 | Elbanizine | −7.1 |
| 64 | Glisindamide | −9.5 |
| 65 | Camicinal | −7.5 |
| 66 | Tegobuvir | −7.6 |
| 67 | Flumeridone | −7.7 |
| 68 | Lomitapide | −7.4 |
| 69 | Mazokalim | −7.8 |
| 70 | Losulazine | −8.3 |
| 71 | Peraquinsin | −7.5 |
| 72 | Leflunomide | −8.6 |
| 73 | Afacifenacin | −7.5 |
| 74 | Ag-13958 | −7.8 |
| 75 | Lorpiprazole | −7.7 |
| 76 | Siponimod | −7.6 |
| 77 | Mespiperone | −7.3 |
| 78 | Xl-019 | −7.7 |
| 80 | Bentipimine | −7.2 |
| 81 | Zosuquidar | −7.5 |
| 82 | Fasitibant | −8.1 |
| 83 | Amg-517 | −8.9 |
| 84 | Ci-988 | −7.7 |
| 85 | Boxidine | −8.0 |
| 86 | Glicaramide | −7.7 |
| 87 | Niaprazine | −7.0 |
| 88 | Isometamidium | −7.2 |
| 89 | Metofenazate | −7.3 |
| 90 | Trenizine | −7.6 |
| 91 | Flotrenizine | −7.5 |
| 92 | Difluanine | −7.4 |
| 93 | Ramatroban | −7.0 |
| 94 | Sonepiprazole | −7.0 |
| 95 | Tafamidis | −7.0 |
| 96 | Verlukast | −7.0 |
| 97 | Ropizine | −7.0 |
| 98 | Lotrifen | −7.0 |
| 99 | Mioflazine | −7.0 |
| 100 | Talotrexin | −7.0 |
| 101 | Tiflamizole | −7.0 |
| 102 | Trefentanil | −7.0 |
| 103 | Asapiprant | −7.0 |
| 104 | Lidoflazine | −7.0 |
| 105 | Ruzadolane | −6.9 |
| 106 | Preladenant | −6.9 |
| 107 | Medibazine | −6.9 |
| 108 | Delavirdine | −6.9 |
| 109 | Piflutixol | −6.9 |
| 110 | Eberconazole | −6.9 |
| 111 | Teneligliptin | −6.9 |
| 112 | Daltroban | −6.9 |
| 113 | Deracoxib | −6.9 |
| 114 | Relenopride | −6.9 |
| 115 | Avagacestat | −6.9 |

### Example 7: Assessment of Functional Activity of MK2 Binding Activators by Western Blot Analysis

In order to assess the functional activity of the putative MK2 binding activators, a series of these compounds (i.e., Leflunomide, Netupitant, Paliperidone, Lomitapide, and Perospirone) were analyzed by Western blot analysis. Rat microvascular endothelial cells were grown to confluency in 35 mm tissue culture dishes. The cells were treated with or without the putative MK2 binding activators at 20 μM concentration for 30 minutes at 37° C. As a positive control, one of the cell dishes was exposed to UV light for ca. 10 minutes. The cells were then lysed with 1% triton solution containing various anti-proteolytic agents and resulting lysates were loaded onto 4-12% acrylamide (Bis-Tris) gels and electrophoresed. The separated protein bands were electro-transferred onto PVDF membranes. The membrane was first blocked in 5% non-fat milk powder and then probed with either rabbit anti-rat phospho-HSP-27 or anti-rat HSP-27, as the normalizing control, followed by the secondary anti-rabbit HRP conjugate for detection. The membrane was washed, treated with a chemiluminescence substrate, and digitally imaged followed by quantification by densitometry. The intensity of detected signal was proportional to the MK2 activation potency of the tested compound.

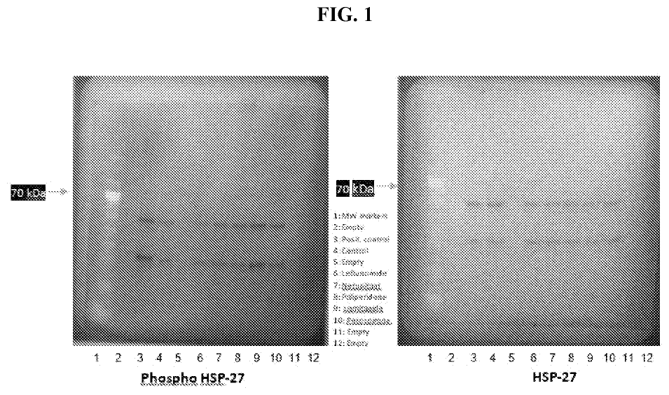

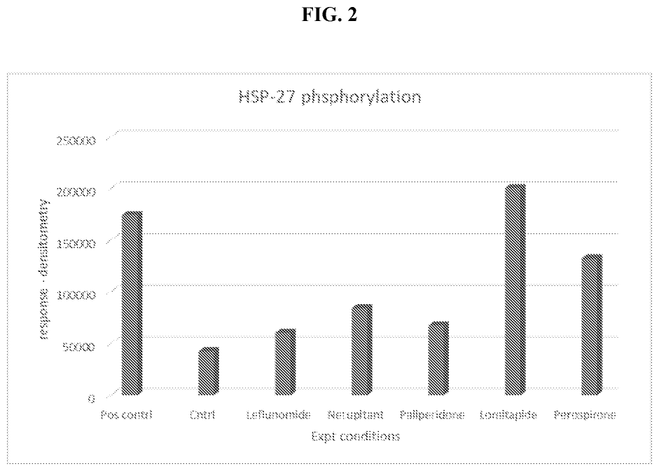

Western blot results from the compounds analyzed by the above-described method are provided in FIG. 1 and graphically as a histogram plot in FIG. 2, after normalization for HSP-27 signal.

The blot image on the left of FIG. 1 (i.e., blot image labelled Phospho HSP-27) displays bands associated with phosphorylated HSP-27, while the blot on the right of FIG. 1 (i.e., blot image labelled HSP-27) shows bands specific for unphosphorylated HSP-27. The two bands seen in each lane of the blots represent monomeric and dimeric forms of both phosphorylated and unphosphorylated HSP-27. The intensity of the bands was quantified by densitometry and then each raw readout was normalized by the signal intensity of unphosphorylated HSP-27, in order to correct for any differences in lysate load across the various treatment conditions. These normalized results are displayed in FIG. 2.

Results plotted in FIG. 2 clearly show >3× increase in phosphorylation above control when cells are stressed by UV irradiation, used as a positive control. Additionally, of the five compounds tested, Lomitapide triggered the strongest increase in HSP-27 phosphorylation.

### Example 8: ELISA MK2 Cell-Based Activity Assay

The MK2 activator candidates, identified using the artificial intelligence (AI)-assisted docking approach, were tested in activity assays for lead selection against the MK2 target. MK2 activation was assessed using (a) a cell-based assay using rat microvascular endothelial cells (RMVEC) and (b) human cancer skin fibroblast cells (Malme-3M). In both assays, an Enzyme-linked ImmunoSorbent Assay (ELISA) technique was used to measure (HSP-27) phosphorylation (DYC2314-2, R&D Systems). Both cell lines were grown to confluency in a 96-well tissue culture plate and treated at 37° C. for 30 minutes with the test compounds at concentrations of 20 μM. The cells were subsequently lysed with a lysis solution containing 0.5% Triton X-100 and anti-proteolysis agents. The resulting lysates were then loaded onto ELISA plates coated with anti-rat phospho-HSP27. After incubation and washing, the plates were treated with an horseradish peroxidase (HRP)-conjugated anti-phospho-HSP27 secondary antibody and then measured for chemiluminescence. Higher activity compounds, acting on MK2, induced higher levels of HSP-27 phosphorylation, as measured by the ELISA.

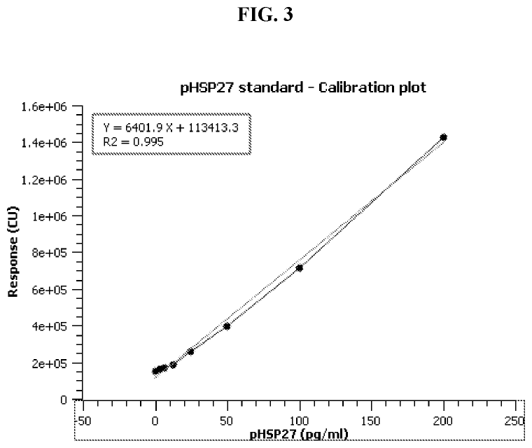

A phospho-HSP27 standard curve for the ELISA, generated by loading standard phospho-HSP27 up to 200 pg/mL, is shown in FIG. 3.

Results from the analysis of some of the AI predicted MK2 activators are shown in Table 4 and Table 5.

TABLE 4
Data from the analysis of a subpopulation of the
predicted MK2 activators using the RMVEC cells

| | Average Response | Ratio |
|---|---|---|
| Compound (20 μM) | HSP27 (pg/mL) | Compound/control |
| Abexinostat (PCI-24781) (t) | 48.4 | 2.3 |
| AC480 (BMS-599626) | 12.1 | 1.3 |
| Adavosertib (MK-1775) (d) | 21.4 | 0.9 |
| AG-13958 (t) | 20.4 | 1.2 |
| AMG-517 (t) | 33.7 | 2.1 |
| AMG-900 (t) | 20.4 | 1.6 |
| Asapiprant | ND | ND |
| AST-487 (NVP-AST487) | 9.0 | 1.3 |
| Avagacestat (BMS-708163) | 5.7 | 0.8 |
| AZD3514 | 5.9 | 0.9 |
| BAF312 (Siponimod) | 6.6 | 1.0 |
| Bafetinib (INNO-406) | 5.8 | 0.8 |
| Bemcentinib (R428) | ND | ND |
| BGT226 (NVP-BGT226) (t) | 19.4 | 1.1 |
| BMS-833923 | 8.8 | 1.3 |
| CC-401 Hydrochloride | 6.3 | 0.9 |
| CEP-32496 | 6.2 | 0.9 |
| Delavirdine (mesylate) (t) | 18.4 | 1.1 |
| Deracoxib (t) | 26.5 | 1.2 |
| Dropropizine | 1.2 | 0.1 |
| ENMD-2076 | 4.6 | 0.3 |
| Flupenthixol dihydrochloride | 10.4 | 1.1 |
| Golvatinib (E7050) | 1.2 | 0.3 |
| GSK1070916 | 0.9 | 0.2 |
| GSK461364 (t) | 39.0 | 1.8 |
| Imidocarb dipropionate | 11.7 | 1.3 |
| Leflunomide | 5.2 | 1.3 |
| Levodropropizine (t) | 47.1 | 2.3 |
| Lomitapide (t) | 39.2 | 3.4 |
| LY2584702 | 0.0 | 0.3 |
| MK2-AP (batch#2) (d) | 11.7 | 2.7 |
| Mocetinostat (MGCD0103) | 17.8 | 1.0 |
| Mubritinib (TAK 165) | 34.0 | 1.6 |
| Nafamostat Mesylate | 11.7 | 1.3 |
| Netupitant | 2.7 | 0.7 |
| Nilotinib (AMN-107) | 18.2 | 1.1 |
| Paliperidone | 4.3 | 1.1 |
| Perospirone hydrochloride (t) | 43.2 | 2.2 |
| PF-3758309 | 4.8 | 1.0 |
| Preladenant | 4.2 | 0.9 |
| Radotinib (t) | 21.7 | 1.0 |
| RAF265 (CHIR-265) | 8.5 | 0.9 |
| Raltitrexed (t) | 25.4 | 1.1 |
| Ramatroban (t) | 27.6 | 1.3 |
| Tafamidis (t) | 18.6 | 0.8 |
| TAK-901 | −0.2 | 0.2 |
| Tegobuvir (d) | 26.2 | 1.2 |
| Teneligliptin hydrobromide | 4.0 | 0.8 |
| XL019 (t) | 15.2 | 0.6 |
| XL228 | 5.7 | 0.4 |
| Ziprasidone HCl (t) | 32.3 | 1.5 |
| Zosuquidar (LY335979) 3HCl | 11.8 | 0.7 |

(t) = compounds analyzed in three separate and independent assays
(d) = compounds analyzed in two separate and independent assays

For compounds with results from multiple assays, the average response is being shown in Table 4.

TABLE 5
Data from the analysis of predicted MK2
activators using the Malme-3M cells

| Compound (20 μM) | Pho-HSP27 (pg/mL) | Ratio to Control |
|---|---|---|
| Control | 22.5 | 1.0 |
| Positive control | 806.0 | 35.8 |
| Lomitapide | 848.5 | 37.7 |
| Perospirone | 720.7 | 32.0 |
| Abexinostat | 836.0 | 37.2 |
| AG13958 | 15.2 | 0.7 |
| Levodropropizine | 621.3 | 27.6 |
| Nilotinib | 12.7 | 0.6 |
| AMG900 | 21.2 | 0.9 |
| AMG517 | 29.9 | 1.3 |
| Delavirdin | 17.3 | 0.8 |
| BGT226 | 26.9 | 1.2 |
| Mocetinostat | 21.2 | 0.9 |
| Tafamidis | 24.1 | 1.1 |
| XL019 | 15.0 | 0.7 |
| Radotinib | 20.8 | 0.9 |
| Ziprasidone | 35.4 | 1.6 |
| Mubritinib | 36.8 | 1.6 |
| Ramatroban | 33.2 | 1.5 |
| Deracoxib | 26.2 | 1.2 |
| Raltitrexed | 38.7 | 1.7 |
| GSK461364 | 22.1 | 1.0 |
| Adavosertib | 15.7 | 0.7 |
| Tegobuvir | 20.9 | 0.9 |

A substantially stronger response was observed with the human skin fibroblast cell line in comparison to the rat endothelial cells. Overall, of the compounds tested, the ones that displayed the most robust response, included Lomitapide, Perospirone, Abexinostat and Levodropropizine.

### INCORPORATION BY REFERENCE

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

We claim:

1. An MK2 activator for treating a vascular disorder or an endothelial barrier disorder in a patient in need thereof, wherein the MK2 activator has a molecular weight of less than 1000 Da; and the MK2 activator binds to a binding pocket located on the C-lobe domain and C-terminal Regulatory Domain of MK2.

2. The MK2 activator of claim 1, wherein the C-lobe domain of MK2 comprises the amino acid sequence of SEQ ID NO: 2.

3. The MK2 activator of claim 1, wherein the C-terminal Regulatory Domain of MK2 comprises the amino acid sequence of SEQ ID NO: 3.

4. The MK2 activator of claims 1-3, wherein the binding pocket located on the C-lobe domain and C-terminal Regulatory Domain of MK2 comprises the amino acid residues M275, K276, I279, V352, E354, E355, S358, A359, and T362.

5. The MK2 activator of claims 1-3, wherein the binding pocket located on the C-lobe domain and C-terminal Regulatory Domain of MK2 comprises the amino acid residues Y260, Y264, S265, N266, H267, E285, F286, P287, N288, P289, E290, and V341.

6. The MK2 activator of claims 1-3, wherein the binding pocket located on the C-lobe domain and C-terminal Regulatory Domain of MK2 comprises the amino acid residues Y260, Y264, S265, N266, H267, A270, I271, S272, P273, G274, M275, K276, R278, I279, E285, F286, P287, N288, P289, E290, V341, E347, R348, E350, D351, V352, E354, E355, S358, A359, and T362.

7. The MK2 activator of claim 1, wherein the binding pocket is located between amino acid residues Y260 and T362.

8. The MK2 activator of claim 1, wherein the binding pocket is located between amino acid residues E290 and D351.

9. The MK2 activator of any one of claims 1-8, wherein the MK2 activator binds to the binding pocket having a binding affinity of about −5 kcal or lower.

10. The MK2 activator of any one of claims 1-9, wherein the MK2 activator is selective for MK2 over other kinases.

11. The MK2 activator of any one of claims 1-10, wherein the MK2 activator is selected from the group consisting of Sepimostat, Bifepramide, Capeserod, Bifeprofen, Lorcinadol, Sulfamazone, Altapizone, Zaldaride, Netupitant, Casopitant, Pipamazine, Saperconazole, Barmastine, Bgt-226, Imidocarb, Elopiprazole, Oxaflumazine, Flupentixol, Clocapramine, Paliperidone, Tiospirone, Sertindole, Perospirone, Ziprasidone, Picloxydine, Amg-548, Cep-32496, Gsk-1070916, Gsk-461364, Ly-2584702, Fludoxopone, Clodoxopone, Amg-900, Mk-6592, Mk-8033, Azd-1775, Mubritinib, Mocetinostat, Pf-3758309, R428, Raltitrexed, XL228, Azd3514, Elinogrel, Tak-901, Chir-265, Bafetinib, Ac-480, Cc-401, Nilotinib, Radotinib, Golvatinib, Abexinostat, Ast-487, Enmd-2076, Bms-833923, Draflazine, Tezosentan, Lometrexol, Cinuperone, Icopezil-maleate, Elbanizine, Glisindamide, Camicinal, Tegobuvir, Flumeridone, Lomitapide, Mazokalim, Losulazine, Peraquinsin, Leflunomide, Afacifenacin, Ag-13958, Lorpiprazole, Siponimod, Mespiperone, XL019, Bentipimine, Zosuquidar, Fasitibant, Amg-517, Ci-988, Boxidine, Glicaramide, Niaprazine, Isometamidium, Metofenazate, Trenizine, Flotrenizine, Difluanine, Ramatroban, Sonepiprazole, Tafamidis, Verlukast, Ropizine, Lotrifen, Mioflazine, Talotrexin, Tiflamizole, Trefentanil, Asapiprant, Lidoflazine, Ruzadolane, Preladenant, Medibazine, Delavirdine, Piflutixol, Eberconazole, Teneligliptin, Daltroban, Deracoxib, Relenopride, Avagacestat, and combinations thereof.

12. The MK2 activator of any one of claims 1-10, wherein the MK2 activator is selected from the group consisting of Sepimostat, Bifepramide, Capeserod, Bifeprofen, Lorcinadol, Sulfamazone, Altapizone, Zaldaride, Netupitant, Casopitant, Pipamazine, Saperconazole, Barmastine, Bgt-226, Imidocarb, Elopiprazole, Oxaflumazine, Flupentixol, Clocapramine, Paliperidone, Tiospirone, Sertindole, Perospirone, Ziprasidone, Picloxydine, Ly-2584702, Fludoxopone, Clodoxopone, Mubritinib, Mocetinostat, Pf-3758309, Raltitrexed, Azd3514, Elinogrel, Ac-480, Abexinostat, Bms-833923, Draflazine, Tezosentan, Lometrexol, Cinuperone, Icopezil-maleate, Elbanizine, Glisindamide, Camicinal, Tegobuvir, Flumeridone, Lomitapide, Mazokalim, Losulazine, Peraquinsin, Afacifenacin, Ag-13958, Lorpiprazole, Siponimod, Mespiperone, Bentipimine, Zosuquidar, Fasitibant, Amg-517, Ci-988, Boxidine, Glicaramide, Niaprazine, Isometamidium, Metofenazate, Flotrenizine, Difluanine, Ramatroban, Sonepiprazole, Tafamidis, Verlukast, Ropizine, Lotrifen, Mioflazine, Talotrexin, Tiflamizole, Trefentanil, Asapiprant, Lidoflazine, Ruzadolane, Preladenant, Medibazine, Delavirdine, Piflutixol, Eberconazole, Teneligliptin, Daltroban, Deracoxib, Relenopride, Avagacestat, and combinations thereof.

13. The MK2 activator of any one of claims 1-10, wherein the MK2 activator is selected from the group consisting of Abexinostat (PCI-24781), Ac-480 (BMS-599626), Adavosertib (MK-1775), Ag-13958, Amg-517, Amg-900, Asapiprant, Ast-487 (NVP-AST487), Avagacestat (BMS-708163), Azd3514, BAF312 (Siponimod), Bafetinib (INNO-406), Bemcentinib (R428), Bgt-226 (NVP-BGT226), Bms-833923, Cc-401 Hydrochloride, Cep-32496, Delavirdine (mesylate), Deracoxib, Dropropizine, Enmd-2076, Flupentixol dihydrochloride, Golvatinib, Gsk-1070916, Gsk-461364, Imidocarb dipropionate, Leflunomide, Levodropropizine, Lomitapide, Ly-2584702, MK2-AP (batch #2), Mocetinostat (MGCD0103), Mubritinib (TAK 165), Nafamostat Mesylate, Netupitant, Nilotinib (AMN-107), Paliperidone, Perospirone hydrochloride, Pf-3758309, Preladenant, Radotinib, RAF265 (Chir-265), Raltitrexed, Ramatroban, Tafamidis, Tak-901, Tegobuvir, Teneligliptin hydrobromide, XL019, XL228, Ziprasidone HCl, Zosuquidar (LY335979) 3HCl, and combinations thereof.

14. The MK2 activator of any one of claims 1-10, wherein the MK2 activator is selected from the group consisting of Lomitapide, Perospirone, Abexinostat, AG-13958, Levodropropizine, Nilotinib, AMG-900, AMG-517, Delavirdin, Bgt-226, Mocetinostat, Tafamidis, XL019, Radotinib, Ziprasidone, Mubritinib, Ramatroban, Deracoxib, Raltitrexed, GSK461364, Adavosertib, Tegobuvir, and combinations thereof.

15. The MK2 activator of any one of claims 1-10, wherein the MK2 activator is selected from the group consisting of Leflunomide, Netupitant, Paliperidone, Lomitapide, Perospirone hydrochloride, and combinations thereof.

16. A composition for the treatment of a vascular disorder or an endothelial barrier disorder comprising a therapeutically effective amount of an MK2 activator of any one of claims 1-15.

17. A pharmaceutical composition for the treatment of a vascular disorder or an endothelial barrier disorder comprising a therapeutically effective amount of an MK2 activator of any one of claims 1-15 and a pharmaceutically acceptable excipient.

18. A unit dosage form for the treatment of a vascular disorder or an endothelial barrier disorder comprising a therapeutically effective amount of an MK2 activator of any one of claims 1-15.

19. A method of treating a vascular disorder or an endothelial barrier disorder in a patient in need thereof, comprising administering to the patient a therapeutically effective amount of an MK2 activator of any one of claims 1-15.

20. The method of claim 19, wherein the vascular disorder is a vascular leak disorder.

21. The method of claim 19, wherein the endothelial barrier disorder is a microvascular barrier leak disorder.

22. The method of claim 21, wherein the microvascular barrier leak disorder is selected from the group consisting of inflammatory bowel disease, brain edema, acute lung injury, acute respiratory distress syndrome, and pulmonary edema.

23. The method of claim 22, wherein the acute respiratory distress syndrome is associated with COVID-19.

24. The method of claim 22 or 23, wherein the acute respiratory distress syndrome is associated with a hyper-permeability of an endothelial barrier.

25. The method of claim 22, wherein the acute respiratory distress syndrome is associated with an infectious disease selected from the group consisting of middle east respiratory syndrome (MERS), severe acute respiratory syndrome (SARS), and pneumonia.

26. The method of claim 22, wherein the acute lung injury is associated with COVID-19.

27. The method of claim 22 or 26, wherein the acute lung injury is associated with a hyper-permeability of endothelial barrier.

28. The method of claim 22, wherein the acute lung injury is associated with an infectious disease selected from the group consisting of middle east respiratory syndrome (MERS), severe acute respiratory syndrome (SARS), and pneumonia.

29. The method of claim 19, wherein the endothelial barrier disorder is a blood vessel barrier leak disorder.

30. The method of claim 29, wherein the blood vessel barrier leak disorder is selected from the group consisting of atherosclerosis, hypertension, preeclampsia, and Kawasaki disease.

31. The method of claim 19, wherein the endothelial barrier disorder is a fibroblast-related disorder.

32. The method of claim 31, wherein the fibroblast-related disorder is selected from the group consisting of wound healing, pulmonary fibrosis, liver fibrosis, vascular fibrosis, kidney fibrosis, and tissue remodeling.

33. The method of claim 19, wherein the vascular disorder is a vascular barrier disorder.

34. The method of claim 33, wherein the vascular barrier disorder is a gastrointestinal disorder.

35. The method of claim 34, wherein the gastrointestinal disorder is associated with COVID-19.

36. The method of claim 34, wherein the gastrointestinal disorder associated with a hyper-permeability of endothelial barrier.

37. A method of treating an acute respiratory distress syndrome in a patient in need thereof, the method comprising administering to the patient a therapeutically effective amount of an MK2 activator of any one of claims 1-15.

38. The method of claim 37, wherein the acute respiratory distress syndrome is associated with COVID-19.

39. A method of treating an acute lung injury in a patient in need thereof, the method comprising administering to the patient a therapeutically effective amount of an MK2 activator of any one of claims 1-15.

40. The method of claim 39, wherein the acute lung injury is associated with COVID-19.

41. The method of any one of claims 19-40, wherein the MK2 activator is administered intravenously to the patient.

42. The method of any one of claims 19-40, wherein the MK2 activator is administered orally to the patient.

43. The method of any one of claims 19-40, wherein the MK2 activator is administered parenterally to the patient.

44. The method of any one of claims 19-43, wherein the patient is a human.


SEQUENCE LISTING (8 sequences)

SEQ ID NO: 1 - protein, 400 aa, Homo sapiens
MLSNSQGQSPPVPFPAPAPPPQPPTPALPHPPAQPPPPPPQQFPQFHVKSGLQIKKNAIIDDYKVTSQVLGLGINGKVLQIFNKRTQEKFALKMLQDCPKARREVELHWRASQCPHIVRIVDVYENLYAGRKCLLIVMECLDGGELFSRIQDRGDQAFTEREASEIMKSIGEAIQYLHSINIAHRDVKPENLLYTSKRPNAILKLTDFGFAKETTSHNSLTTPCYTPYYVAPEVLGPEKYDKSCDMWSLGVIMYILLCGYPPFYSNHGLAISPGMKTRIRMGQYEFPNPEWSEVSEEVKMLIRNLLKTEPTQRMTITEFMNHPWIMQSTKVPQTPLHTSRVLKEDKERWEDVKEEMTSALATMRVDYEQIKIKKIEDASNPLLLKRRKKARALEAAALAH

SEQ ID NO: 2 - protein, 187 aa, Artificial Sequence
  MK2 peptide fragment
LDGGELFSRIQDRGDQAFTEREASEIMKSIGEAIQYLHSINIAHRDVKPENLLYTSKRPNAILKLTDFGFAKETTSHNSLTTPCYTPYYVAPEVLGPEKYDKSCDMWSLGVIMYILLCGYPPFYSNHGLAISPGMKTRIRMGQYEFPNPEWSEVSEEVKMLIRNLLKTEPTQRMTITEFMNHPWIMQ

SEQ ID NO: 3 - protein, 73 aa, Artificial Sequence
  MK2 peptide fragment
STKVPQTPLHTSRVLKEDKERWEDVKEEMTSALATMRVDYEQIKIKKIEDASNPLLLKRRKKARALEAAALAH

SEQ ID NO: 4 - protein, 6 aa, Artificial Sequence
  MK2 peptide fragment
RGDQAF

SEQ ID NO: 5 - protein, 24 aa, Artificial Sequence
  MK2 peptide fragment
TSHNSLTTPCYTPYYVAPEVLGPE

SEQ ID NO: 6 - protein, 9 aa, Artificial Sequence
  MK2 peptide fragment
SNHGLAISP

SEQ ID NO: 7 - protein, 30 aa, Artificial Sequence
  MK2 peptide fragment
YARAAARQARAHPRNPARRTPGTRRGAPAA

SEQ ID NO: 8 - protein, 46 aa, Artificial Sequence
  MK2 peptide fragment
DKERWEDVKEEMTSALATMRVDYEQIKIKKIEDASNPLLLKRRKKA